(19) United States
(12) Patent Application Publication
(10) Pub. No.: US 2022/0175988 A1
(43) Pub. Date: Jun. 9, 2022

(54) PRINTABLE ULTRAVIOLET-C (UV-C) CHAMBER TO DECONTAMINATE REUSABLE PERSONAL PROTECTIVE EQUIPMENT (PPE)

(71) Applicant: The Rockefeller University, New York, NY (US)

(72) Inventors: Alexander Epstein, New York, NY (US); Thomas P. Sakmar, Wading River, NY (US); Thomas Huber, New York, NY (US)

(73) Assignee: The Rockefeller University, New York, NY (US)

(21) Appl. No.: 17/542,685

(22) Filed: Dec. 6, 2021

Related U.S. Application Data
(60) Provisional application No. 63/122,530, filed on Dec. 8, 2020.

(51) Int. Cl. A61L 2/10

(52) U.S. Cl. CPC A61L 2/10; A61L 2202/11; A61L 2202/14; A61L 2202/122

(57) ABSTRACT

A UV-C decontamination apparatus is used to decontaminate PPEs, such as respiratory masks. The apparatus is formed from a 3D printing process which makes the manufacture and use widespread. The apparatus includes a 3D printed chamber and a lid for enclosing the chamber. At least one UV-C lamp is supported in the chamber. Activation of the lamp is designed to decontaminate the mask supported in the chamber from the lid. An electronic switch assembly permits activation of the lamp only upon locking closure of a lid to the chamber. In a further embodiment, the hook is rotatable on the lid so as to rotate the mask in the chamber upon lamp activation.

### FIELD OF THE INVENTION

This application claims priority to U.S. Provisional Patent Application No. 63/122,530, filed on Dec. 8, 2020, the contents of which are incorporated herein by reference in its entirety.

The present invention is directed to a 3D printed UV-C chamber for decontaminating personal protective equipment (PPEs), more particularly, paper/fiber masks such as N95 respiratory masks using UV illumination.

### BACKGROUND OF THE INVENTION

It has long been commercially known to use ultraviolet-C (UV-C) light to decontaminate the surfaces of various medical devices, tools and other objects. Typically decontamination is performed in boxes or chambers equipped with high intensity UV-C lamps. Due to the recent COVID-19 pandemic, the need for such decontamination chambers or boxes for various types of personal protection equipment (PPEs) has become overwhelming. The need is especially acute with respect to the decontamination of respiratory masks, such as N95 masks, the demand for which has greatly increased.

UV-C boxes and chambers currently commercially available are not generally cost-efficient for the decontamination of N95 masks, particularly in a setting where COVID-19 exposure is prevalent, such as hospitals, medical offices and other point of care locations. While devices to decontaminate N95 masks in hospitals are available, the devices are large and expensive and difficult to assemble.

It is desirable to provide a point-of-care UV-C chamber which effectively decontaminates N95 masks, is cost effective to manufacture and easy to use.

### SUMMARY OF THE INVENTION

The present invention provides a UV-C decontamination apparatus which includes an 3D printable chamber having a cylindrical wall, a closed bottom and an open top. The chamber defines an interior for accommodating, for example, an N95 mask. A removable 3D printable lid is provided for closing the open end of the chamber. A plurality of activatable UV-C lamps are supported within the chamber. Activation of the lamps is designed to decontaminate the N95 mask accommodated within the chamber. An electronic switch assembly permits activation of the UV-C lamps only upon locking closure of the lid on the open end of the chamber.

Components of the apparatus are designed to be manufactured using standard 3D printing devices which are cost effective and readily available.

The cylindrical configuration of the chamber allows for maximum internal reflectance and minimal shadowing. To enhance the reflective characteristics, the inside of the chamber is coated with UV-C reflective material.

The arrangement of the UV-C lamps within the chamber is designed to deliver proper UV-C irradiance so as to decontaminate a single N95 mask in a relatively short period of time.

In order to reduce the chance of UV-C exposure to the user, an electronic switch assembly is employed. The UV-C lamps can only be activated once the lid is properly locked onto the open upper end of the chamber. Viewing windows are provided in the lid, positioned over the lamps, so that the UV-C lamp operation can be viewed. The viewing windows are opaque to UV-C light and transparent to visible light.

The chamber is mounted on a base which supports the electronics and an LED indicator light.

In a further embodiment, the N95 may be mounted for rotation within the chamber.

### BRIEF DESCRIPTION OF THE DRAWINGS

FIG. 1 is a top perspective showing of a UV-C decontamination apparatus of the present invention.

FIG. 2 is a top plan view of the UV-C decontamination apparatus in FIG. 1 with the lid removed.

FIG. 3 is a top perspective view of a decontamination chamber of the apparatus in FIG. 1 with the lid removed.

FIG. 4 is a partial vertical sectional showing of the decontamination apparatus in FIG. 1 including an N95 mask supported therein.

FIG. 5 is a plan view of the undersurface of the lid of the decontamination apparatus in FIG. 1.

FIG. 6 is a side plan view of the lid of FIG. 5 supporting the N95 mask.

FIG. 7 shows, in partial section, the lid engaged with the chamber.

FIG. 8 shows, in partial section, the lid and chamber including a magnetic reed switch assembly.

FIG. 9 is a top prospective view of a further embodiment of the apparatus of the present invention.

FIG. 10 is a partial sectional showing of the apparatus in FIG. 9 including an N95 mask supported therein.

FIG. 11 is a top plan view of the apparatus in FIG. 9.

### DETAILED DESCRIPTION OF THE PREFERRED EMBODIMENT

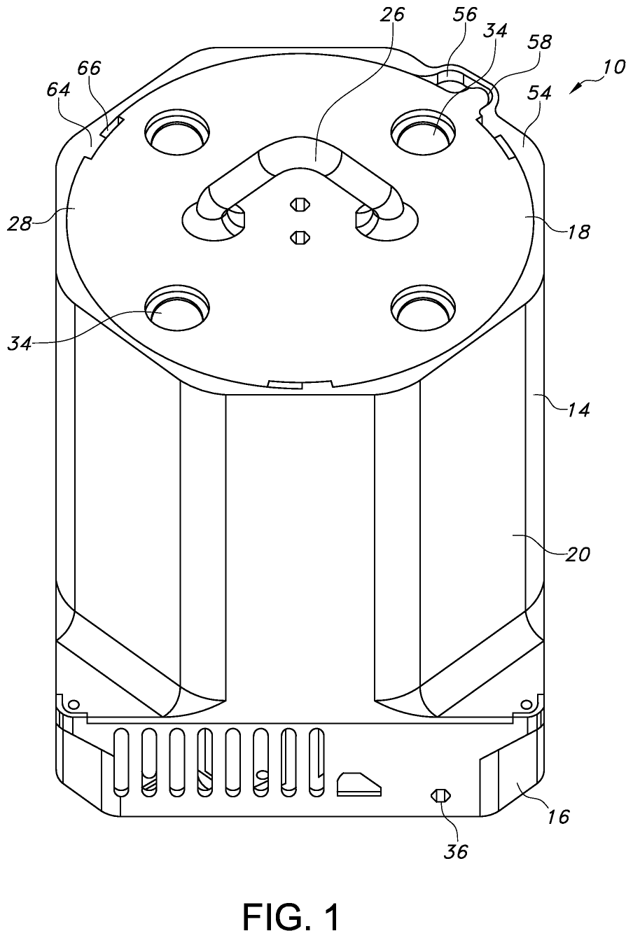

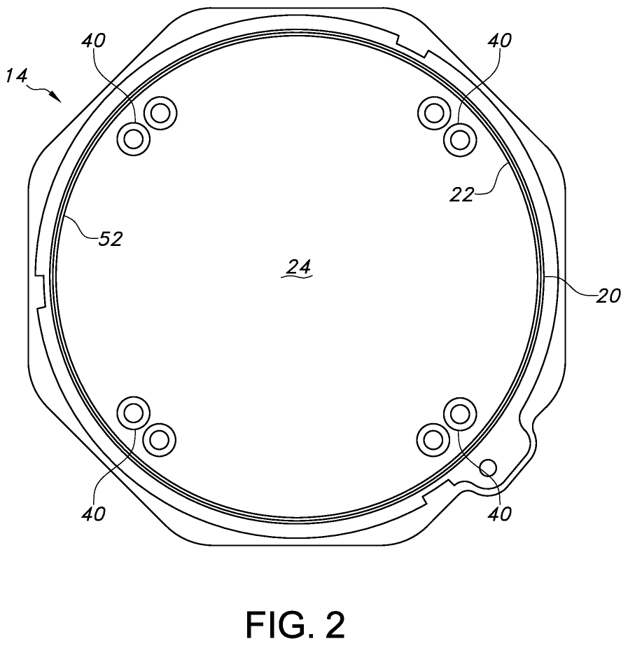

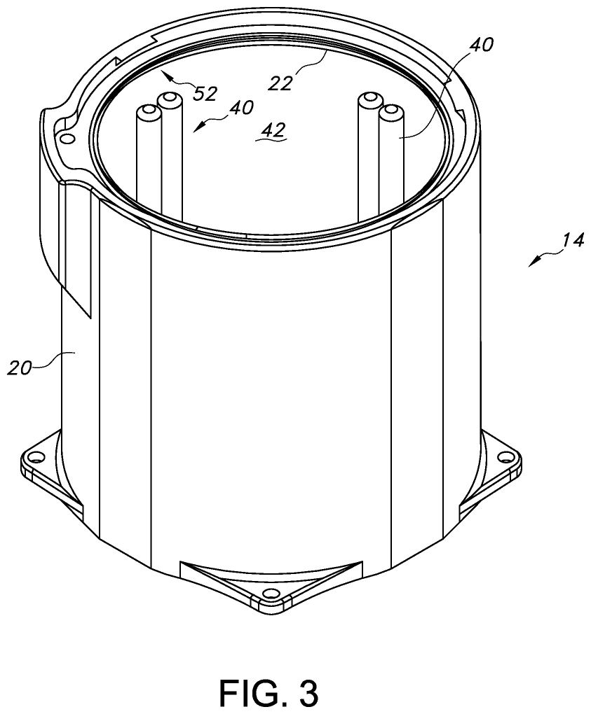

A first embodiment of an apparatus 10 for the decontamination of (PPEs) such as respiratory masks, more particularly, N95 masks 12 is shown in FIGS. 1-8 of the present invention. The invention is particularly useful in decontaminating paper or fiber PPEs which cannot be washed or disinfected with liquid agents. While the invention will be described with regard to respiratory masks, more particularly, N95 masks, the invention may be used on other suitable PPEs.

Referring specifically to FIGS. 1-3, apparatus 10 includes a chamber 14, a base 16 for supporting chamber 14, and a cover or lid 18. The chamber includes a generally cylindrical wall 20 having a cylindrical interior surface 22 and a bottom wall 24.

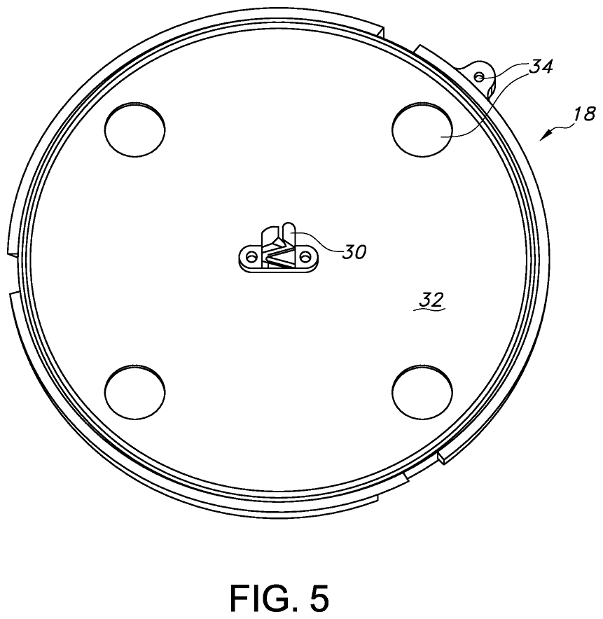

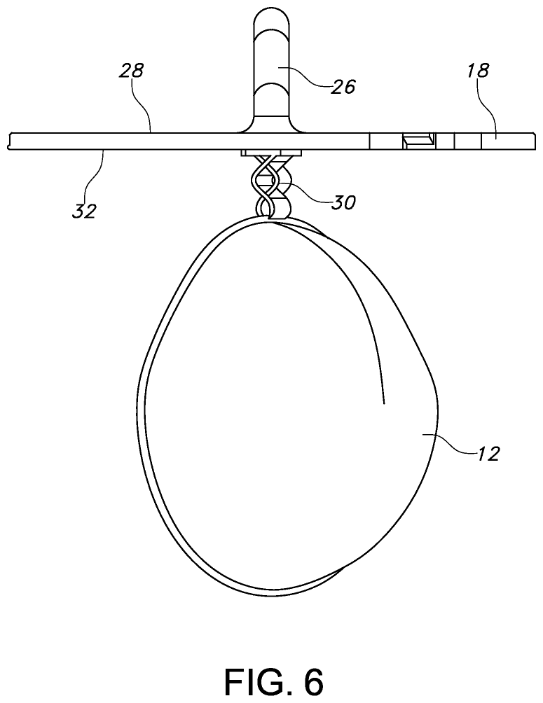

The lid 18, more fully shown in FIGS. 5 and 6, is generally a circular disk-like planar member having a handle 26 on an upper exterior surface 28 and a hook 30 extending from an opposed interior surface 32. A plurality of viewing windows 34, in this case four, extends through lid 18 from the interior surface 32 to the exterior surface 28. The viewing windows 34 as well as the hook 30 will be described in further detail below.

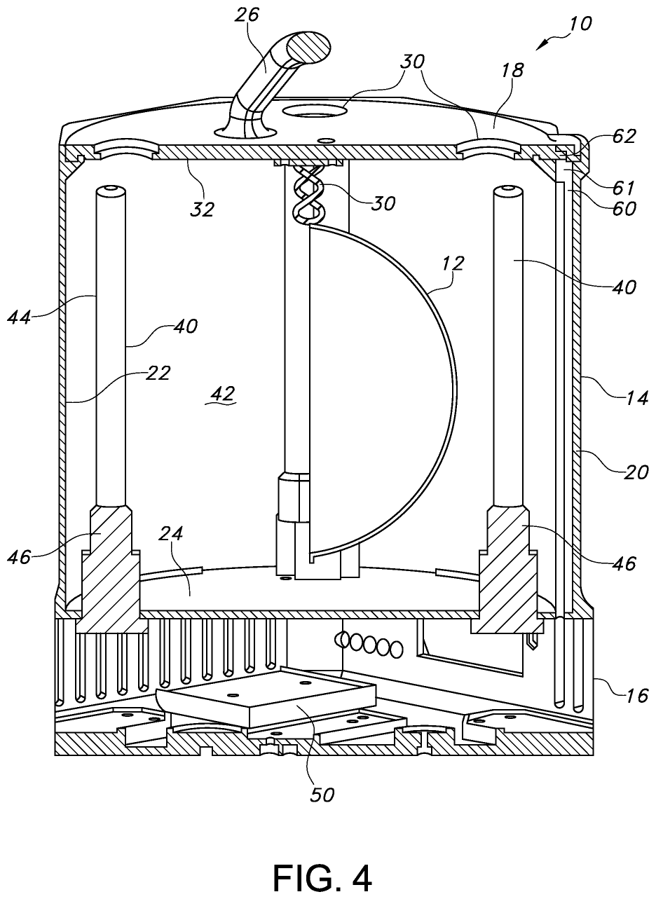

The chamber 14 is mounted on a generally rectangular base 16, which as shown in FIG. 4 houses a plurality of electronic components 50 which will be described in further detail below.

The chamber 14, base 16, lid 18 and hook 30 of apparatus 10 may all be formed from a 3D printable matrix by standard 3D printing techniques using a 3D printer. Recent advances in 3D printing technology have resulted in more effective and portable 3D printers thus enabling their widespread use. The printers can be maintained at a low cost and can produce three-dimensional items, such as the apparatus of the present invention, at virtually any location. Thus, 3D printing techniques enable the apparatus of the present invention to be produced on an as needed basis on a wide scale. The apparatus can therefore be made available quickly and easily to point-of-use/care location, such as hospitals, medical offices, clinics, firehouses, etc. where there is a need to decontaminate N95 masks on a small scale.

Referring additionally to FIG. 4, a plurality of UV-C lamps 40 are supported within the interior 42 of chamber 14. As used herein the term UV-C lamp or lamp is understood to refer to conventional UV-C bulbs, UV-C light emitting diodes and any suitable UV-C radiant energy generating devices which are well known to those skilled in the art. The UV-C lamps 40 are conventionally known and are generally elongate, tubular and U-shaped in configuration. Each lamp 40 is electrically connected at one end by a receptacle 46 extending from the bottom wall 24 of chamber 14. The UV-C lamps 40 are arranged preferably adjacent the interior wall 22 of chamber 14. The lamps 40 are electrically connected to electronic components 50 in the base 16 shown schematically in FIG. 4 which provide for activation and deactivation of UV-C lamps 40.

The lid 18 is constructed and arranged so that when properly positioned over the open end 52 of chamber 14, the viewing windows 34 generally align over the spaced apart lamps 40. In that regard, the viewing windows are formed of a material which is opaque to UV-C light, but transparent to visible light. As the UV-C lamps emit a small amount of visible light, a user can observe and confirm the activation of the UV-C lamps through the viewing window. This is particularly useful where the 3D printable matrix forming the components is dark in color. However, where the 3D printable matrix is white, the material itself is translucent and may “glow” from the visible UV-C light.

As more fully shown in FIGS. 4, 5 and 6, the interior surface 32 of lid 18 includes hook 30 centrally mounted and extending therefrom. The hook 30 permits proper hanging securement of the N95 mask 12 in a central location with respect thereto. As the lid 18 is positioned over the open end 52 of chamber 14, the N95 mask extends into the interior 42 of chamber 14 in position where the UV-C lamps 40 surround the N95 mask. Thus positioned, sufficient UV-C illumination is evenly distributed to the N95 mask to effectively decontaminate the mask supported therein. It is contemplated that with the current embodiment a single N95 mask can be decontaminated in a relatively short time period, about 2½ minutes. This is a substantially shorter decontamination time then with currently commercially available devices.

The interior wall 22 and bottom wall 24 of chamber 14 as well as the interior surface 32 of lid 18 are coated with a UV-C reflective sheet or membrane, preferably PTFE film, so that that the complete decontamination of the N95 mask is enhanced. Also, the shape of the chamber being generally cylindrical allows for maximum internal reflection and minimal shadowing. Unlike other polymer films, it has been found that PTFE film is highly reflective of UV-C light. This allows for a more simplified construction eliminating the need for structural reflectors.

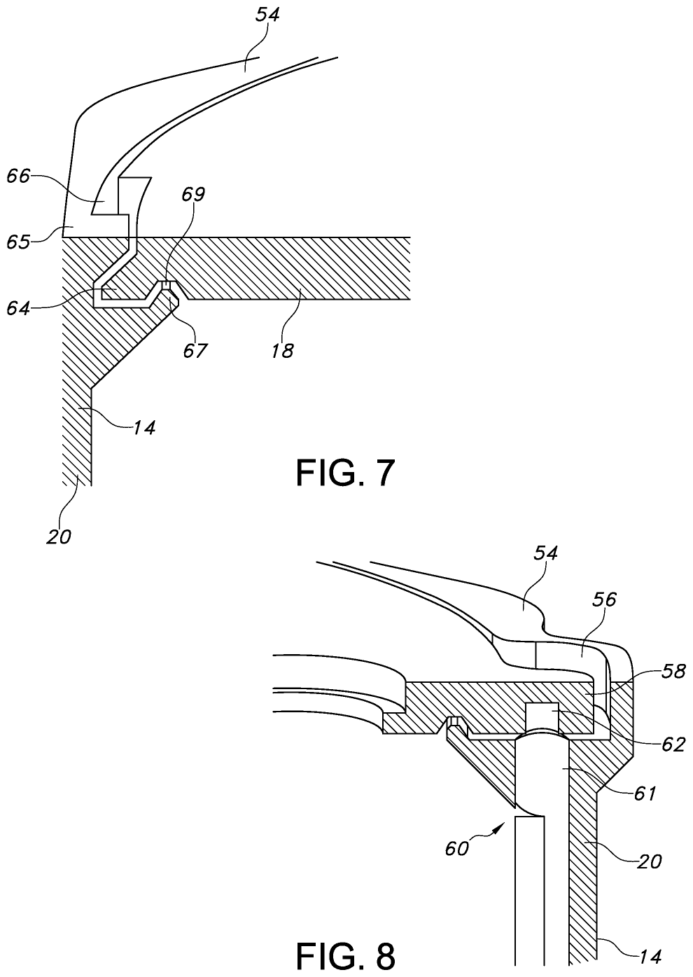

Referring more fully now to FIGS. 7 and 8, the apparatus of the present invention provides an electronic reed switch assembly 60 which assures that the lid 18 is properly positioned over the open end 52 of chamber 14 and locks to the chamber before UV-C lamp activation can occur. The switch assembly 60 includes a magnetic reed switch 61 supported by the chamber 14 and a magnet 62 supported in lid 18.

As additionally shown in FIGS. 1 and 8, the upper edge 54 of chamber 14 includes a recess 56 which accommodates an extending tab 58 which extends from the perimeter of lid 18. The cylindrical wall 20 supports at the upper edge of chamber 14, adjacent at the recess 56, reed switch 61. The lid 18 supports and contains the magnet 62. The reed switch assembly 60 is a conventional switching assembly which permits switching or activation between an “on” and “off” position. As is well known to those skilled in the art, the reed switch contains a pair of magnetic flexible reeds separated by a gap in the open/off position. The reeds are moved into contact or “on” position by activation of a magnetic field. Other forms of reed switches as well as other switches may also be employed.

When the lid 18 is positioned over the open end 52 of chamber 14, the lid 18 must be rotated to a proper position so that the magnet 62 is positioned adjacent the reed switch 61 so as to activate the reed switch assembly thereby permitting activation of the UV-C lamps 40.

Referring additionally to FIGS. 1 and 7, an arrangement of locking recesses and locking tabs is also provided between the lid 18 and the chamber 14 to assure that when the reed switch assembly 60 is activated, the lid 18 is locked onto the chamber preventing removal. The upper edge 54 of chamber 14 includes a pair of edge tabs 64 spaced about the circumference. Lid 18 includes a pair of edge recesses 66 designed for alignment with edge tabs 64. As more fully shown in FIG. 7, each edge tab 64 is designed to lock into the corresponding recess 66 when the lid 18 is properly positioned. As the lid 18 is rotated, the edge tabs 64 slide under a lip 65 adjacent recess 66. An engaging guide arm 67 on the upper edge 54 of chamber 14 rides in a guide undercut 69 of lid 18 to assure locking engagement of the lid 18 to chamber 14. In only this position will the reed switch assembly 60 be activated. The lid 18 can only be removed by rotating the cover to disengage the edge tabs from the edge recesses which thereby also disengages the reed switch assembly so as to assure deactivation of the UV lamps.

Electronic activation of the reed switch assembly 60 is provided in conventional fashion by electronics 50 housed within base 16. In addition, when the reed switch assembly 60 is engaged and the UV-C lamps are activated, the indicator LED 36 is illuminated providing additional indication that the UV-C lamps are activated.

The apparatus is driven by an original prototype printed circuit board (prototype PCB), which is custom-designed for this purpose. It incorporates an Arduino-based microcontroller which receives input from the reed switch. The microcontroller sends output to a solid-state relay on the prototype PCB, which activates and deactivates the lamps. The microcontroller also sends output to an LED and a piezoelectric speaker to provide feedback to the device user.

The software for the device performs the following functions: 1) ensures that the lamps are always deactivated when the reed switch is open (in the “off” position), 2) decontaminates the device for the proper time interval when the lid is closed, 3) sends appropriate feedback to the user at the beginning and end of decontamination cycles, and 4) immediately deactivates the lamps and sounds an alarm if the decontamination cycle is terminated prematurely by opening the lid.

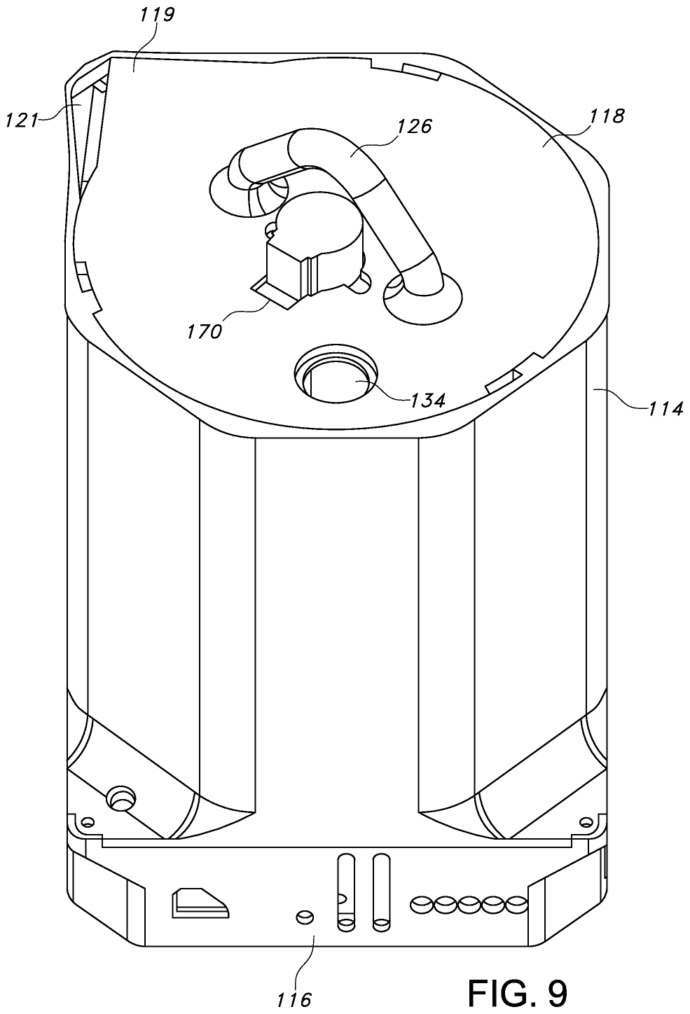

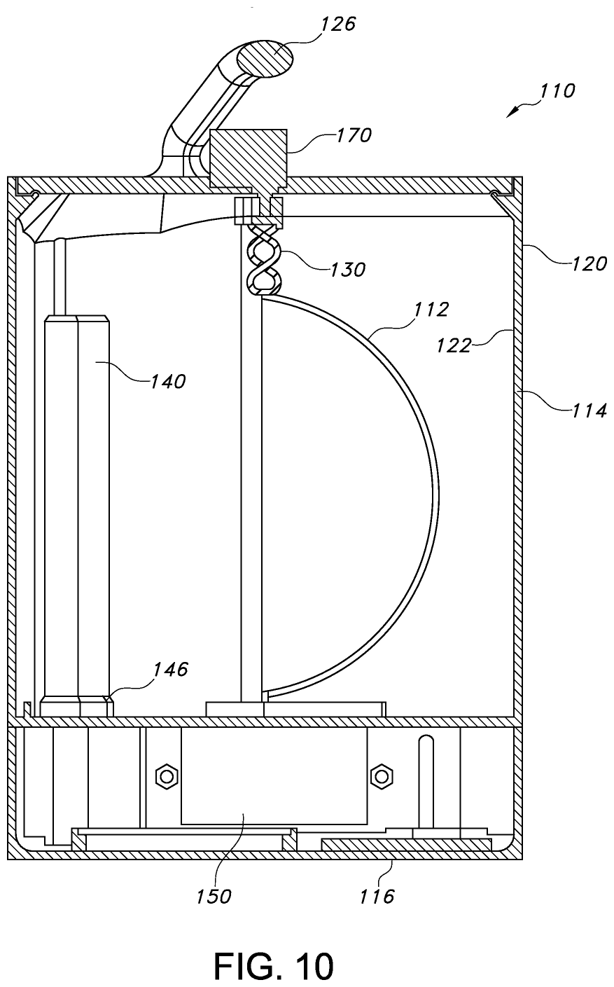

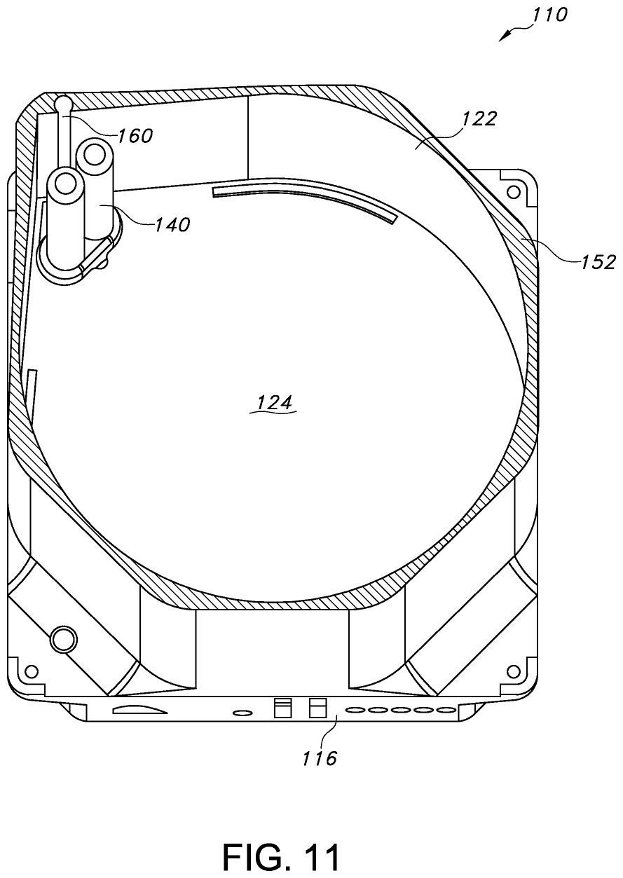

A further embodiment of the present invention is shown in FIGS. 9-11. Like reference numerals will be used to denote like elements.

Apparatus 110 is similar to that described above. Apparatus 110 includes a chamber 114, a base 116, a cover or lid 118 and hook 130 which also may be formed from a 3D printable matrix by standard 3D printing techniques using a 3D printer. In the present embodiment, the apparatus 110 employs a single UV-C lamp 140 of the type described above, positioned adjacent the interior wall 124 of chamber 114. As with the above, the lamp 140 is electronically connected through a receptacle 146 to electronic components 150 in the base 116 which provides for activation of the lamp 140.

In the present embodiment, the chamber 114 has a generally cylindrical wall 120 but is formed to include a teardrop extension 121. The single UV-C lamp 140 is positioned adjacent the teardrop extension 121. This positioning as well as the geometry of the extension 121 provides enhanced reflection of the UV-C light into the interior of chamber 114 and onto the N-95 mask 112 supported therein.

The lid 118 also includes a teardrop protrusion 119 and employs a single viewing window 134, similar to the window described above, which is generally aligned with the lamp 140 when the lid is positioned at the open end 152 of chamber 114.

As with the above embodiment, lid 118 includes a hook 130 centrally mounted and extending from an interior surface 132 thereof.

In the present embodiment, hook 130 is connected to a stepper motor 170 which extends through the upper surface 128 beneath handle 126. The stepper motor 170 is rotationally positioned on the lid 118 so that it can rotate the hook 130 and thereby the N-95 mask 112 within the interior of chamber 114.

The stepper motor 170 is connected by an electrical wire or cable (not shown) extending outside chamber 140 to electronics 150 supported in base 116 which power the motor. The stepper motor 170 is activated upon activation of the UV-C lamps 140. In the present embodiment, a prototype PCB includes a ULN2003 stepper motor driver and controls the motor that rotates the mask. The motor is turned on when the mask is being decontaminated, and off when decontamination is complete.

As with the above described embodiment, the interior wall 122 and bottom wall 124 of chamber 114 as well as the interior surface (not shown) of lid 118 all include a PTFE reflective membrane thereon.

Apparatus 110 allows for complete decontamination of the N-95 112 mask using only a single UV-C lamp 140. This arrangement allows for the apparatus 110 to be smaller, more inexpensively constructed and more easily implemented. One trade off is that the decontamination process takes a longer period of time, approximately 4 times as long as the above-described embodiment having multiple lamps.

In all other respects, the apparatus 110 operates similar to apparatus 10 described above, including the locking engagement of the lid 118 to chamber 114, which only allows lamp activation upon such locking engagement. A magnetic reed structure 160 is employed in a manner similar to that described above.

Various changes to the foregoing described and shown structures would now be evident to those skilled in the art. Accordingly, the particularly disclosed scope of the invention is set forth in the following claims.

What is claimed is:

1. A UV-C decontamination apparatus for decontamination of a respiratory mask, said apparatus comprising:
a 3D printed chamber having a generally cylindrical wall, a closed bottom end and an open top end; said chamber defining an interior for insertably accommodating said N95 mask;
a 3D printed removable lid for closing said open end;
at least one activatable UV-C lamp arranged within said interior of said chamber extending from a bottom wall and adjacent an interior wall, activation of said at least one lamp designed to decontaminate said mask accommodated within said chamber interior; and
an electronic switch assembly which permits activation of said UV-C lamp only upon locking closure of said lid.

2. An apparatus of claim 1 wherein said lid includes at least one viewing window for viewing the interior of said chamber.

3. An apparatus of claim 1 wherein said at least one UV-C lamp is position adjacent the interior wall of said cylindrical wall.

4. An apparatus of claim 1 wherein said electronic switch assembly includes a magnetic reed switch assembly.

5. An apparatus of claim 1 wherein said bottom wall and said interior wall of said chamber and an underside of said lid are coated with a UV-C reflective membrane.

6. An apparatus of claim 2 further including a plurality of UV-C lamps and a plurality of said viewing windows and wherein said lamps are circumferentially spaced about the interior wall and said viewing windows in said lid are arranged to be positioned over said lamps.

7. An apparatus of claim 1 further including a 3D printed hook positioned on an undersurface of said lid, said hook designed to support said mask within said chamber interior.

8. An apparatus of claim 6 wherein said viewing windows are opaque to UV-C light and transparent to visible light.

9. An apparatus of claim 4 further including a base for supporting said chamber; said base having electronic components for activating said UV-C lamp and said magnetic read switch assembly.

10. An apparatus of claim 9 further including an LED indicator light on said base for indicating UV-C lamp activation.

11. An apparatus of claim 4 wherein said magnetic reed switch assembly includes a reed switch supported by said cylindrical wall of said chamber and a magnet supported by said lid, movement of said lid with respect to said chamber effecting activation/deactivation of said reed switch assembly.

12. An apparatus of claim 11 wherein an upper edge of said cylindrical wall and a perimeter of said lid includes interlocking tabs and recesses to assure locking closure of said lid on said chamber when said magnetic reed switch assembly is activated.

13. An apparatus of claim 5 wherein said UV-C membrane is PTFE.

14. An apparatus of claim 1 wherein said generally cylindrical wall and said lid include a tear drop extension and wherein said at least one activatable lamp is positioned adjacent said tear drop extension of said cylindrical wall.

15. An apparatus of claim 14 wherein said hook is rotatably mounted to said lid for rotating said mask in said chamber.

16. An apparatus of claim 15 wherein said lid supports a stepper motor for effecting rotation of said hook.

17. An apparatus of claim 1 wherein said mask is an N95 mask.

18. A method of decontaminating a respiratory mask comprising the steps of:
providing a 3D printed chamber having a generally cylindrical wall, a closed bottom end, an open upper end defining a chamber interior, said chamber having at least one activatable UV-C lamp adjacent the interior of said cylindrical wall;
providing a 3D printed lid for enclosing said open upper end;
providing an electronic switch assembly which permits activation of said UV-C lamp only upon locking attachment of said lid to said open upper end of said chamber;
attaching said mask to an undersurface of said lid;
locking said lid to said open upper end of chamber to position said mask within the interior of the chamber; and
activating said UV-C lamp to decontaminate said mask within said chamber.

19. A method of claim 18 wherein said lid includes a 3D printed hook on an undersurface of said lid and wherein said attaching step includes hanging the mask on said hook.

20. A method of claim 18 wherein said lid further includes a viewing window adjacent said UV-C lamp, and further including the step of:
viewing visible light through said viewing window for confirming activation/deactivation of the UV-C lamp.

21. A method of claim 20 wherein said electronic switch assembly includes a magnetic reed switch assembly.

22. A method of claim 21 wherein said reed switch assembly includes a reed switch supported by said chamber and a magnet supported by said lid, and further including the step of:
rotating said lid to said locked position to active said magnetic reed switch assembly.

23. A method of claim 18 wherein said generally cylindrical wall and said lid include a tear drop extension and said at least one activatable lamp is positioned adjacent said tear drop extension of said chamber.

24. A method of claim 23 wherein said hook is rotatably supported by said lid and further includes the step of:
rotating said hook to cause rotation of said mask within the interior of said chamber.

25. A method of claim 23 wherein said lid supports a stepper motor for causing said rotation of said hook.

26. A method of claim 17 wherein said mask is an N95 mask.
